(19) United States
(12) Patent Application Publication
(10) Pub. No.: US 2025/0127407 A1
(43) Pub. Date: Apr. 24, 2025

(54) MULTI-OBJECT BIOMETRIC INFORMATION MEASUREMENT DEVICE, SYSTEM FOR DETECTING ABNORMAL SIGNS OF DISEASE INCLUDING THE SAME, AND METHOD THEREOF

(71) Applicant: ELECTRONICS AND TELECOMMUNICATIONS RESEARCH INSTITUTE, Daejeon (KR)

(72) Inventors: Jae Young JUNG, Daejeon (KR); You Jin KIM, Daejeon (KR); Kwang Hyo CHUNG, Daejeon (KR); Seng Kyoun JO, Daejeon (KR)

(21) Appl. No.: 18/921,210

(22) Filed: Oct. 21, 2024

(30) Foreign Application Priority Data
Oct. 20, 2023 (KR) 10-2023-0141273

(51) Int. Cl. A61B 5/0205; A61B 5/00; A61B 5/01; A61B 5/024; A61B 5/05; A61B 5/08; A61B 90/00; G06T 7/00; G06T 7/20; G06V 10/25; G16H 50/20

(52) U.S. Cl. CPC A61B 5/0205; A61B 5/0059; A61B 5/01; A61B 5/05; A61B 5/7275; A61B 90/06; G06T 7/0012; G06T 7/20; G06V 10/25; G16H 50/20; A61B 5/024; A61B 5/0816; A61B 2090/061; A61B 2560/0252; A61B 2562/0271; G06T 2207/20084; G06T 2207/30004; G06V 2201/07

(57) ABSTRACT

Provided is a multi-object biometric information measurement device, which includes at least one sensor module and a processor connected to the sensor module, and the processor recognizes at least one of sneezing information, heart rate/respiratory rate information and core temperature information of an object located in a measurement target space based on sensing data collected through the sensor module.

### CROSS-REFERENCE TO RELATED APPLICATION

This application claims priority to and the benefit of Korean Patent Application No. 10-2023-0141273, filed on Oct. 20, 2023, the disclosure of which is incorporated herein by reference in its entirety.

### BACKGROUND

### 1. Field of the Invention

The present invention relates to a multi-object biometric information measurement device that enables early detection of abnormal signs of disease of multiple objects, a system for detecting abnormal signs of disease including the same, and a method thereof.

### 2. Description of Related Art

Recently, due to highly contagious, high-risk, high-mortality respiratory infectious diseases such as coronavirus disease (COVID-19), Middle East respiratory syndrome (MERS), and severe acute respiratory syndrome (SARS), normal social functions are paralyzed and massive social, economic, and human damage is occurring domestically and internationally. Respiratory infectious diseases such as the above diseases mainly occur in indoors, which are closed spaces/crowded places/close-contact settings (hereinafter referred to as “3Cs”), and it is most important to block the spread of infectious diseases in advance through early diagnosis of abnormal signs of disease of multiple objects in indoor spaces. The presence or absence of abnormal signs of respiratory infectious diseases is determined through initial symptoms such as fever, coughing, sneezing, and difficulty in breathing, and constant monitoring of these initial symptoms is required.

Conventionally, thermal imaging cameras capable of measuring body temperature remotely and in a non-contact manner were mainly used to detect initial symptoms of respiratory infectious diseases. However, when measuring the body temperature of people passing by thermal imaging cameras installed in a waiting room in an airport, school, hospital, or bus terminal, the accuracy of measuring skin surface temperature is reduced due to the influence of external temperature, and the sensitivity of the remote thermal imaging cameras is low, and thus there is a high possibility of missing patients with a fever. In addition, there is a problem with the conventional method that it is not possible constantly monitor the body temperature of many people in the 3Cs.

In addition, conventional thermal imaging cameras also have the problem of being insufficient in preemptively responding to the spread of infection because they do not have the function to analyze symptoms such as coughing and sneezing that accompany fever and heart rate/respiratory rate.

### SUMMARY OF THE INVENTION

The present invention is directed to providing a multi-object biometric information measurement device that enables early detection of abnormal signs of disease of multiple objects of multiple objects in the 3Cs (closed spaces/crowded places/close-contact settings), a system for detecting abnormal signs of disease including the same, and a method thereof. According to an aspect of the present invention, there is provided a multi-object biometric information measurement device including at least one sensor module, and a processor connected to the sensor module, in which the processor recognizes at least one of sneezing information, heart rate/respiratory rate information, and core temperature information of an object located in a measurement target space based on sensing data collected through the sensor module.

In some embodiments of the present invention, the sensor module may include a first image sensor that acquires a thermal image of the measurement target space, a second image sensor that acquires a real image of the measurement target space, a distance measurement sensor that measures a distance to the object, and an environmental sensor that measures an atmospheric air temperature of the measurement target space.

In some embodiments of the present invention, the processor may include an object detection unit that detects an object corresponding to a preset class in a thermal image acquired through a first image sensor and a real image acquired through a second image sensor, and a biometric information recognition unit that recognizes biometric information including at least one of sneezing information, heart rate/respiratory rate information, and core temperature information of the object detected by the object detection unit.

In some embodiments of the present invention, the processor may further include an image correction unit that corrects the thermal image and the real image so that the images match, and the object detection unit may generate a bounding box of the detected object to perform object tracking when the object corresponding to the preset class is present in the corrected real image.

In some embodiments of the present invention, the biometric information recognition unit may recognize the sneezing information by applying the detected object to a behavior detection model to determine whether the object is coughing/sneezing, and counting the number of times the object coughs/sneezes while tracking the object that coughs or sneezes.

In some embodiments of the present invention, the biometric information recognition unit may detect a specific region in the detected object, acquire a radiant heat measurement value of the specific region, and calculate a core temperature of the object based on the radiant heat measurement value.

In some embodiments of the present invention, the biometric information recognition unit may calculate the core temperature by applying a compensation value set according to a distance between a distance measurement sensor and an object to the radiant heat measurement value to correct the radiant heat measurement value and applying the corrected radiant heat measurement value, an atmospheric air temperature measured through an environmental sensor, and a head circumference of an object to the Biot number.

In some embodiments of the present invention, the biometric information recognition unit may recognize a heart rate and respiratory rate of the object by analyzing a micro-Doppler signal, which is a signal generated when an antenna radiation beam formed by a distance measurement sensor is reflected by the object and returned.

In some embodiments of the present invention, the processor may further include a metadata generation unit that generates biometric information including at least one of the recognized sneezing information, heart rate/respiratory rate information, and core temperature information of the object, and device state information of the multi-object biometric information measurement device as metadata, and transmits the generated metadata to an external device through a communication module.

In some embodiments of the present invention, the processor may further include an abnormal-signs-of disease detection unit that detects abnormal signs of disease of multiple objects within the measurement target space based on the biometric information recognized by the biometric information recognition unit, and predicts a disease occurrence risk.

According to another aspect of the present invention, there is provided a system for detecting abnormal signs of disease including a plurality of multi-object biometric information measurement devices, each of which is installed in one measurement target space and recognize at least one type of biometric information among sneezing information, heart rate/respiratory rate information, and core temperature information of an object that is present in the corresponding measurement target space, and transmit metadata including the biometric information and device state information to a management server, and a management server that predicts a disease occurrence risk based on the metadata from the plurality of multi-object biometric information measurement devices.

In another aspect of the present invention, the management server may generate notification information including the predicted disease occurrence risk, and notify users of the notification information.

According to still another aspect of the present invention, there is provided a method of detecting abnormal signs of disease including receiving, by a processor, sensing data including at least one of a thermal image, a real image, distance measurement information, and an ambient temperature from a sensor module, detecting, by the processor, an object corresponding to a preset class in the thermal image and the real image, and recognizing, by the processor, biometric information including at least one of sneezing information, heart rate/respiratory rate information, and core temperature information of the detected object.

In some embodiments of the present invention, in the detecting of the object, the processor may correct the thermal image and the real image so that the images match and generate a bounding box of the detected object to perform object tracking when the object corresponding to the preset class is present in the corrected real image.

In some embodiments of the present invention, in the recognizing of the biometric information, the processor may recognize the sneezing information by applying the detected object to a behavior detection model to determine whether the object is coughing/sneezing, and counting the number of times the object coughs/sneezes while tracking the object that coughs or sneezes.

In some embodiments of the present invention, in the recognizing of the biometric information, the processor may detect a specific region in the detected object, acquire a radiant heat measurement value of the specific region, and calculate a core temperature of the object based on the radiant heat measurement value.

In some embodiments of the present invention, in the recognizing of the biometric information, the processor may calculate the core temperature by applying a compensation value set in the distance measurement information and an object to the radiant heat measurement value to correct the radiant heat measurement value and applying the corrected radiant heat measurement value, an atmospheric air temperature measured through an environmental sensor, and a head circumference of an object to the Biot number.

In some embodiments of the present invention, in the recognizing of the biometric information, the processor may recognize a heart rate and respiratory rate of the object by analyzing a micro-Doppler signal, which is a signal generated when an antenna radiation beam formed by a distance measurement sensor is reflected by the object and returned.

In some embodiments of the present invention, the method may further include generating, by the processor, metadata including the biometric information and device state information and transmitting the metadata to a management server, after the recognizing of the biometric information.

In some embodiments of the present invention, the method may further include detecting, by the processor, abnormal signs of disease of multiple objects within a measurement target space based on the recognized biometric information recognized and predicting a disease occurrence risk, after the recognizing of the biometric information.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a diagram showing a system for detecting abnormal signs of disease according to an embodiment of the present invention;

FIG. 2 is an exemplary diagram for describing the spatial installation and operation of a multi-object biometric information measurement device according to an embodiment of the present invention;

FIG. 3 is a block diagram for describing the multi-object biometric information measurement device according to the embodiment of the present invention;

FIG. 4 is a block diagram for describing a function of the processor illustrated in FIG. 3;

FIG. 5 is an exemplary diagram for describing the Biot number concept for calculating core temperature according to an embodiment of the present invention;

FIG. 6 is a diagram for describing a method of detecting abnormal signs of disease according to an embodiment of the present invention;

FIG. 7 is a flowchart for describing a method of recognizing the biometric information of multiple objects according to an embodiment of the present invention; and

FIG. 8 is a flowchart for describing a method of calculating core temperature according to an embodiment of the present invention.

### DETAILED DESCRIPTION OF EXEMPLARY EMBODIMENTS

Hereinafter, a multi-object biometric information measurement device, a system for detecting abnormal signs of disease including the same, and a method thereof according to an embodiment of the present invention will be described in detail with reference to the accompanying drawings. In this process, the thickness of the lines and the sizes of the components illustrated in the drawings may be exaggerated for the sake of clarity and convenience of description. In addition, the terms described below are terms defined in consideration of their functions in the present invention, and may vary depending on the intention or custom of the user or operator. Therefore, the definitions of these terms should be based on the contents of the entire specification.

Recently, domestically and internationally, highly contagious, high-risk, high-mortality infectious diseases with strong transmissibility, such as coronavirus disease (COVID-19), Middle East respiratory syndrome (MERS), and severe acute respiratory syndrome (SARS), have caused enormous damage. These diseases mainly occur indoors, which are the 3Cs (closed spaces/crowded places/close-contact settings), and it is most important to prevent the diseases through early diagnosis of abnormal signs of disease of multiple objects in indoor spaces.

Therefore, the present invention proposes a technology that uses information and communications technology (ICT) to detect abnormal signs of respiratory disease of multiple objects in the 3Cs early and centrally manages the results on a cloud-based service platform so that the risk of abnormal signs of disease can be provided nationwide.

To this end, the present invention may provide early detection of the risk of abnormal signs of disease and alarm services for the risk of abnormal signs of disease by continuously, precisely, and efficiently measuring the biometric information of multiple objects in the 3Cs (closed spaces/crowded places/close-contact settings) using the multi-object biometric information measurement device. Here, the biometric information may include sneezing information, heart rate/respiratory rate information, core temperature information, etc. of the object.

In addition, the present invention enables the use of a nationwide network alarm service that provides early detection of the risk of abnormal signs of disease and the risk of abnormal signs of disease to all citizens by performing data monitoring, risk degree assessment, service management, etc. on a cloud-based service platform for abnormal object radiant heat measurement values and abnormal object behavior detection results collected from the multi-object biometric information measurement devices installed nationwide.

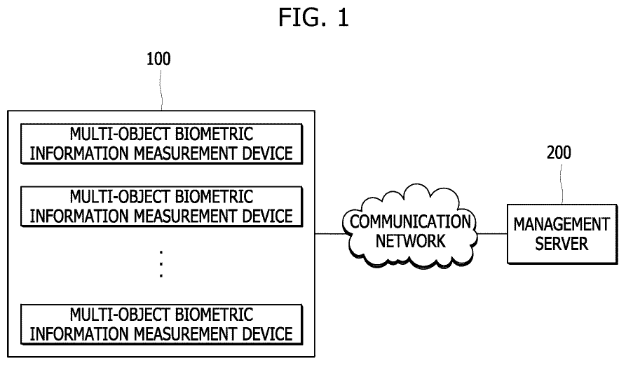

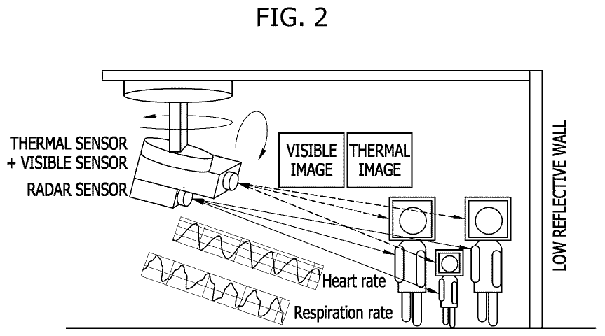

FIG. 1 is a diagram showing a system for detecting abnormal signs of disease according to an embodiment of the present invention, and FIG. 2 is an exemplary diagram for describing the spatial installation and operation of a multi-object biometric information measurement device according to an embodiment of the present invention.

Referring to FIG. 1, the system for detecting abnormal signs of disease according to an embodiment of the present invention includes a plurality of multi-object biometric information measurement devices 100 and a management server 200.

Each of the plurality of multi-object biometric information measurement devices 100 is installed in one measurement target space, may recognize at least one type of biometric information among sneezing information, heart rate/respiratory rate information, and core temperature information of an object that is present in the corresponding measurement target space, and may transmit metadata including the recognized biometric information and device state information to the management server 200. Here, the measurement target space may be the 3Cs (closed spaces/crowded places/close-contact settings), but the scope of the present invention is not limited thereto, and the multi-object biometric information measurement device 100 according to the present invention may be installed in various types of spaces.

The multi-object biometric information measurement device 100 may be installed on a ceiling in the 3Cs as shown in FIG. 2, and may continuously recognize a thermal image, real image (visible light) image, radiant heat, heart rate, respiratory rate, core temperature, etc. of multiple objects while rotating up, down, left, and right through a motion controller (not shown). Therefore, the multi-object biometric information measurement device 100 has a feature of being able to measure the biometric information of multiple people at the same time so that the installation costs of the multi-object biometric information measurement device can be reduced.

The multi-object biometric information measurement device 100 may measure the biometric information of multiple objects in the measurement target space in a non-contact manner.

The management server 200 may receive metadata from the plurality of multi-object biometric information measurement devices 100 and store the received metadata in a database (not shown). In this case, the management server 200 may receive the metadata through an upload folder server program and store the metadata in the database.

The management server 200 may detect abnormal signs of disease of multiple objects within the measurement target space at an early stage based on the metadata from the plurality of multi-object biometric information measurement devices 100.

In addition, the management server 200 may predict the disease risk or disease occurrence risk based on the metadata from the plurality of multi-object biometric information measurement devices 100. In this case, the management server 200 may apply at least one of the sneezing information, heart rate/respiratory rate information, and core temperature information to a disease prediction model to predict the disease risk (disease occurrence risk). In this case, the management server 200 may predict the disease risk (disease occurrence risk) by space, region, or nationwide, etc.

The management server 200 may generate notification information based on the metadata received from the plurality of multi-object biometric information measurement devices 100 and notify users of the generated notification information. That is, the management server 200 may generate notification information including a region with a high disease risk (e.g., a location of multi-object biometric information measurement devices 100 with a high disease risk), the disease occurrence risk, etc., and transmit the generated notification information to users of the corresponding region and users of the entire region. In this case, the management server 200 may transmit the notification information in various forms such as text messages, emails, and SNS services.

In addition, an abnormal-signs-of disease detection platform is installed on the management server 200, and the abnormal-signs-of disease detection platform may predict the disease occurrence risk, etc. through a prediction model and provide an alarm service based on the prediction result.

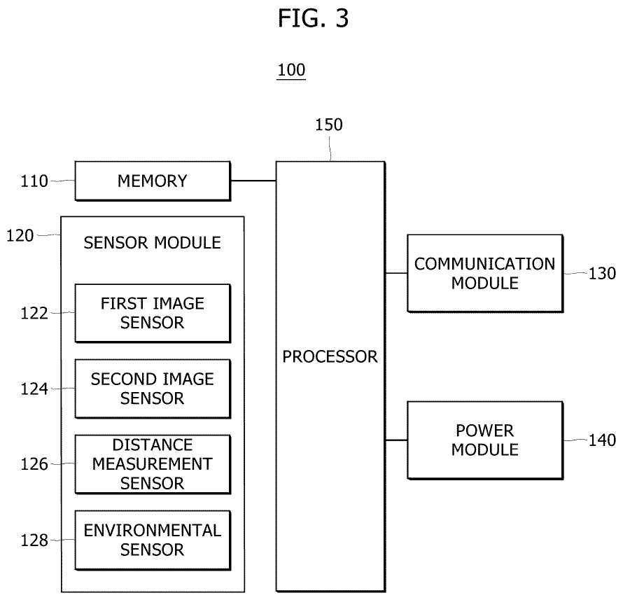

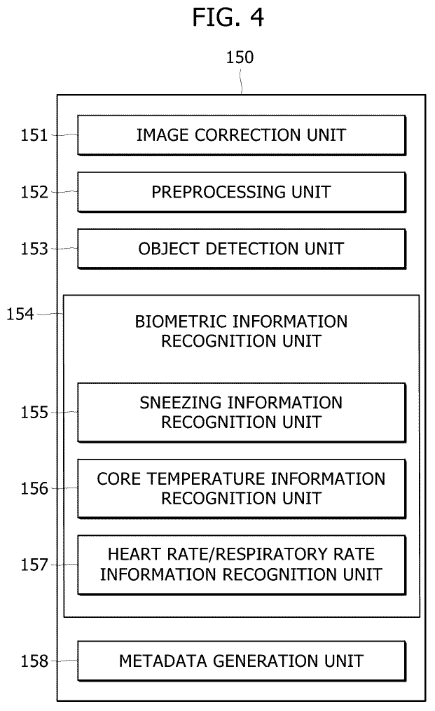

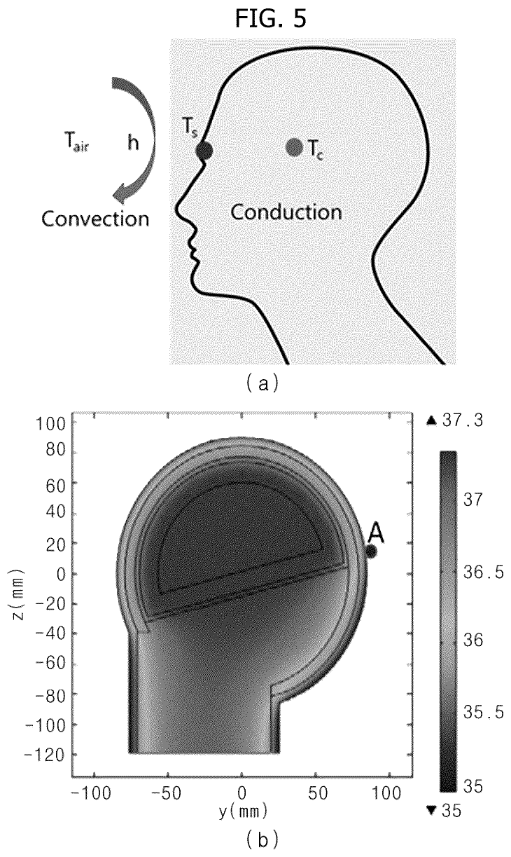

FIG. 3 is a block diagram for describing a multi-object biometric information measurement device according to an embodiment of the present invention, FIG. 4 is a block diagram for describing a function of the processor illustrated in FIG. 3, and FIG. 5 is an exemplary diagram for describing the Biot Number concept for calculating core temperature according to an embodiment of the present invention.

Referring to FIG. 3, the multi-object biometric information measurement device 100 according to an embodiment of the present invention includes a memory 110, a sensor module 120, a communication module 130, a power module 140, and a processor 150.

The memory 110 is a component that stores data related to the operation of the multi-object biometric information measurement device 100. In particular, a program that (application or applet) that allows at least one of sneezing information, heart rate/respiratory rate information, and core temperature information of an object located in the measurement target space to be recognized based on sensing data may be stored in the memory 110, and the stored information may be selected by the processor 150 as needed. That is, various types of data generated during the execution of an operating system or program (application or applet) for driving the multi-object biometric information measurement device 100 are stored in the memory 110. In this case, the memory 110 collectively refers to a non-volatile storage device that maintains stored information even when power is not supplied and a volatile storage device that requires power to maintain stored information. In addition, the memory 110 may perform a function of temporarily or permanently storing data processed by the processor 150. Here, in addition to the volatile storage device that requires power to maintain stored information, the memory 110 may include magnetic storage media or flash storage media, but the scope of the present invention is not limited thereto.

The sensor module 120 may acquire sensing data such as a thermal image, a real image, distance measurement information, and an ambient temperature for the measurement target space and transmit the images and information to the processor 150.

The sensor module 120 may include a first image sensor 122, a second image sensor 124, a distance measurement sensor 126, and an environmental sensor 128.

The first image sensor 122 may acquire a first image of the measurement target space and transmit the acquired first image to the processor 150. Here, the first image may be a thermal image. The first image sensor 122 may be implemented as, for example, a thermal image sensor. The thermal image sensor may detect thermal radiation emitted from each of at least one subject, and output the detected body temperature in various colors through thermal resolution. The thermal image may include a temperature distribution image. Therefore, the first image sensor 122 may be used to measure the radiant heat of multiple objects in a non-contact manner within the measurement target space.

The second image sensor 124 may acquire a second image of the measurement target space and transmit the acquired second image to the processor 150. Here, the second image may be a real image. The second image sensor 124 may be implemented as, for example, a visible light sensor, etc. The second image sensor 124 may be used to detect the coughing/sneezing, inner canthus, etc. of multiple objects located in the measurement target space.

Hereinafter, for the convenience of description, the first image will be referred to as a thermal image and the second image will be referred to as a real image.

The distance measurement sensor 126 may measure a distance to an object and transmit the measured distance measurement information to the processor 150. In addition, the distance measurement sensor 126 may transmit a micro-Doppler signal, which is a signal generated when an antenna radiation beam is reflected by an object and returned, to the processor 150. The micro-Doppler signal may be used to measure the heart rate/respiratory rate of the object located in the measurement target space. The distance measurement sensor 126 may be implemented as an ultrasonic sensor, a radar sensor, a LiDAR sensor, etc.

The environmental sensor 128 may measure the atmospheric temperature, wind speed (air flow), etc. of the measurement target space and transmit the measured atmospheric temperature and wind speed to the processor 150. The environmental sensor 128 may be implemented as a temperature sensor, a wind speed sensor, a humidity sensor, etc.

The communication module 130 is built into the multi-object biometric information measurement device 100 or is connected thereto through a connector, and may provide a communication interface required to provide transmission and reception signals between the multi-object biometric information measurement device 100 and the management server 200 in the form of packet data by linking with a communication network. In particular, the communication module 130 may transmit metadata generated by the processor 150 to the management server 200. This communication module 130 may be implemented in various forms that enable communication in the field without an Internet network, such as a wireless communication module such as LTE, low power wide region (LPWA), or a mobile communication module.

The power module 140 may supply power to the communication module 130, the sensor module 120, and the processor 150. In addition, the power module 140 may be implemented as a battery, etc.

The processor 150 may be configured to control the overall operation of the multi-object biometric information measurement device 100. For example, the processor 150 may execute software (e.g., a program) stored in the memory 110 to control at least one component among the components connected to the processor 150 (e.g., the memory 110, sensor module 120, communication module 130, and power module 140). The processor 150 may be implemented as an application specific integrated circuit (ASIC), a digital signal processor (DSP), a programmable logic device (PLD), a field programmable gate array (FPGA), a central processing unit (CPU), a microcontroller, and/or a microprocessor, but is not limited thereto.

The processor 150 may recognize at least one of sneezing information, heart rate/respiratory rate information, and core temperature information of the object located in the measurement target space based on sensing data collected through the sensor module 120. Here, the sensing data may include a thermal image, a real image, distance measurement information, ambient temperature (atmospheric air temperature), etc.

In addition, the processor 150 may generate metadata including biometric information including at least one of the sneezing information, heart rate/respiratory rate information, and core temperature information of the object and device state information of the multi-object biometric information measurement device 100, and transmit the generated metadata to the management server 200 through the communication module 130.

The processor 150 may include an image correction unit 151, a preprocessing unit 152, an object detection unit 153, a biometric information recognition unit 154, and a metadata generation unit 158.

The image correction unit 151 may correct the thermal image acquired through the first image sensor 122 and the real image acquired through the second image sensor 124 so that the images match.

Since the first image sensor 122 and the second image sensor 124 have different physical characteristics such as sensors and lenses for each product, the resolution, field of view (FOV), location of the center point of view, etc. thereof are different. Therefore, in order to match the object location, it is necessary to match the thermal image of the first image sensor 122 and the real image of the second image sensor 124.

Accordingly, the image correction unit 151 may perform image matching by adjusting the intrinsic parameters of the first image sensor 122 and the second image sensor 124 so that the positions of objects in the thermal image and the real image match. Here, the intrinsic parameters may include the resolution, field of view, sensor size, focal length, distortion rate, etc.

That is, the image correction unit 151 may perform image matching by using the intrinsic parameters used when expressing the coordinate systems of the first image sensor 122 and the second image sensor 124. In this case, the image correction unit 151 may use the calibrateCamera function provided by Open Source Computer Vision Library (OpenCV) that calculates the coordinates of corners of a chess board image to extract the intrinsic parameters of each of the first image sensor 122 and the second image sensor 124. Therefore, the image correction unit 151 may adjust the intrinsic parameters of the first image sensor 122 and the second image sensor 124, such as the field of view and resolution, through the calibrateCamera function to match the thermal image and the real image. Through the image matching, the object location in the thermal image and the object location in the real image may be matched.

The preprocessing unit 152 may perform preprocessing on the real image corrected by the image correction unit 151. The preprocessing unit 152 may perform a function of adjusting the number of frames per second (fps) of the input video according to a change in features (movement) of a frame, a function of converting a size of the frame input for object recognition, a data augmentation function for supplementing insufficient training data, etc. on the real image collected in real time. In order to increase the accuracy of an object detection algorithm, various types of training data are required, and data augmentation may be a technique widely used in the object detection algorithm.

The object detection unit 153 may detect an object corresponding to a preset class (e.g., a person) in the corrected or preprocessed real image. In this case, the object detection unit 153 may detect a person by applying an object detection algorithm to the corrected or preprocessed real image.

In addition, when the object corresponding to the preset class is present in the corrected or preprocessed real image, the object detection unit 153 may generate a bounding box of the detected object. The bounding box of the object may be used to recognize the biometric information of the object.

The object detection unit 153 may apply an image recognition technique to the corrected or preprocessed real image to automatically detect an object that appears in the corresponding image, determine whether the object is present, and calculate an object distribution. The object detection unit 153 may perform object detection and object tracking by applying a deep learning model to the corrected or preprocessed real image. The object detection unit 153 may detect and track not only one object but also a plurality of objects (multiple objects) at the same time. The object detection unit 153 may calculate the object distribution (distribution of the number of objects) according to a measurement direction based on the object detection result. The object detection unit 153 may transmit a command to a driving unit (not shown) to detect an object, so that the driving unit may rotate the sensor module 120 according to the command, and the sensor module 120 transmit a thermal image or a real image generated while rotating to the object detection unit 153.

The biometric information recognition unit 154 may recognize at least one type of biometric information among the sneezing information, heart rate/respiratory rate information, and core temperature information of the object detected by the object detection unit 153.

The biometric information recognition unit 154 may include a sneezing information recognition unit 155, a core temperature information recognition unit 156, and a heart rate/respiratory rate information recognition unit 157.

The sneezing information recognition unit 155 may recognize the sneezing information of an object detected by the object detection unit 153 by applying the object to a deep learning model to determine whether the object is coughing/sneezing, and counting the number of times the object coughs/sneezes while tracking the object that coughs or sneezes. Here, the sneezing information may include the location of the object, the number of times the object coughs, the number of times the object sneezes, etc.

For example, the sneezing information recognition unit 155 may recognize the sneezing information by tracking a location of the object that coughs or sneezes and the number of times the object coughs or sneezes using a coughing/sneezing recognition model (behavior detection model) generated through a supervised learning technique based on a convolutional neural network (CNN).

The core temperature information recognition unit 156 may detect a specific region of an object detected by the object detection unit 153, acquire a radiant heat measurement value of the specific region, and recognize the core temperature information of the object based on the radiant heat measurement value. Hereinafter, the specific region may include, for example, an inner canthus region.

Hereinafter, a method by which the core temperature information recognition unit 156 recognizes the core temperature information will be described in detail.

The core temperature information recognition unit 156 may detect a face region from the object detected by the object detection unit 153, extract the inner canthus region from the detected face region, and acquire a radiant heat measurement value of the extracted inner canthus region.

That is, the core temperature information recognition unit 156 may detect the face region from the object using a face detection algorithm.

Then, the core temperature information recognition unit 156 may extract the inner canthus region from the detected face region. The inner canthus region is the inside corner of the eye where the upper and lower eyelids meet, has thin skin, is less exposed to the environment, and is located right above the major artery, and thus it is known through many documents as the most suitable location for detecting a fever. In this case, detection of the inner canthus region of the object may be performed by either a thermal image sensor or a visible light sensor, or by a combination of the two sensors.

When the inner canthus region is extracted, the core temperature information recognition unit 156 may acquire the radiant heat measurement value of the inner canthus region from the images by applying an image recognition technique to the thermal image and the real image. The acquired radiant heat measurement value acquired at this time may have an error due to the distance between the thermal image sensor and the object.

Therefore, the core temperature information recognition unit 156 may compensate the radiant heat measurement value based on distance data between the distance measurement sensor 126 and the object (or the inner canthus of the object). That is, the core temperature information recognition unit 156 may correct (compensate) the radiant heat measurement value by applying a compensation value determined in advance according to the distance. Since a temperature measurement value of an object (target) of the same size generally decreases as the distance between the thermal image sensor and the object increases, compensation of the measurement value is necessary. For example, the core temperature information recognition unit 156 may compensate the radiant heat measurement value in such a way that, by setting the reference distance to 2 m, 1 degree is added to the radiant heat measurement value when the distance to the object is 3 m, and 2 degrees is added to the radiant heat measurement value when the distance to the object is 4 m.

Meanwhile, in order to detect abnormal signs of disease of the object, it is more effective to use the core temperature than the skin temperature. The radiant heat measurement value refers to the skin temperature, and the skin temperature is different from the core temperature. Accordingly, the core temperature is needed to detect abnormal signs of disease of the object.

Therefore, the core temperature information recognition unit 156 of the present invention may calculate the core temperature using the corrected radiant heat measurement value. In this case, the core temperature information recognition unit 156 may calculate the core temperature by applying the Biot Number concept.

The core temperature information recognition unit 156 may calculate the core temperature by applying the object's head circumference, atmospheric air temperature, radiant heat measurement value, convection coefficient, and the object's thermal conductivity to the Biot number. That is, the core temperature information recognition unit 156 may calculate the core temperature using the following Equation 1.

$$\frac{kh}{L} = \frac{{T}_{c} - {T}_{s}}{{T}_{s} - {T}_{a}} \qquad [ Equation 1 ]$$

Here, h may refer to the convection coefficient, k may refer to the thermal conductivity, and L may refer to the head circumference/3 of the object, Tₛ may refer to the skin temperature (radiant heat measurement value), Tₐ may refer to the atmospheric air temperature, and T_{c} may refer to the core temperature. The convection coefficient and the thermal conductivity may be predefined values, the head circumference may be a value measured by applying an image analysis technique to the face region of the object, and the atmospheric air temperature may be a temperature measured through the environmental sensor 128. The thermal conductivity k may be applied as a value of 0.3 [W/m° C.] in the case of human skin. The L value may vary depending on the size of the human head.

For reference, the Biot number is a measure of the degree of a temperature drop between the surface and the inside of an object, and may be expressed as a ratio of convective heat transfer and internal heat conduction as in Equation 2 below.

$$Biot Number = \frac{convection outside the object}{conduction inside the object} = \frac{h ( Ts - Tair )}{\frac{k ( Tc - Ts )}{L}} \qquad [ Equation 2 ]$$

Here, h is the convection coefficient. In the case of air, it has a value of about 5 to 25 [W/m²K] in natural convection, and has a wide range of about 20 to 300 [W/m²K] in the case of forced convection. The difference between forced convection and natural convection lies in the movement of the fluid. Forced convection is the movement of the fluid due to the application of an external force, while natural convection is the movement of the fluid due to a density difference. In the case of natural convection, since the fluid moves due to a density difference caused by a temperature difference, when the temperature difference is not large, the movement of the fluid is slow and the heat transfer is relatively slow. On the other hand, in the case of forced convection, since the fluid moves due to an external force (e.g., the wind from an electric fan), the movement of the fluid is faster and the heat transfer is more efficient. k is the thermal conductivity, and it is known to have a value of 0.3 [W/m° C.] for human skin. L refers to a characteristic length and is defined as volume/region. When the human head is considered to be a sphere, L may be defined as (head circumference/3) and varies depending on the size of the human head. Tₛ refers to a measured skin temperature, Tₐᵢᵣ refers to atmospheric air temperature, and T_{c} refers to core temperature, as shown FIG. 5(a).

When the Biot number is 0, it is an ideal state where the temperature inside the object is uniform, and when the Biot number is less than 0.1, it may be assumed that the temperature inside the object is sufficiently uniform. In the present invention, it is assumed that the temperature inside the human head is uniform, and the correlation between the measured temperature of the inner canthus and the core temperature is analyzed. FIG. 5(b) shows the difference between the forehead temperature and the core temperature based on a normal subject. It can be seen that the temperature around the head is the same in blue, and the temperature inside the head shows a uniform distribution. In this case, if the difference between the skin temperature and the core temperature is calculated and compensated, an error between the skin temperature and the internal core temperature may be corrected.

Accordingly, the core temperature information recognition unit 156 may calculate the core temperature using the Biot number.

The heart rate/respiratory rate information recognition unit 157 may recognize the heart rate/respiratory rate information by interpreting a micro-Doppler signal, which is a signal generated when a radar signal is emitted to an object and is reflected by the object, to analyze the heart rate and respiratory rate of the object. The heart rate/respiratory rate information recognition unit 157 may analyze the heart rate and respiratory rate of the object by analyzing the micro-Doppler signal, which is a signal generated when an antenna radiation beam formed by the distance measurement sensor 126 is reflected by an object and returned. Here, the micro-Doppler signal may be a signal with which periodic vibration (breathing, respiratory rate) information that may be detected from the front, side, and back of the object may be interpreted in the frequency domain.

As described above, the biometric information recognition unit 154 may recognize biometric information including at least one of the sneezing information, heart rate/respiratory rate information, and core temperature information of the object.

The metadata generation unit 158 may generate the biometric information including at least one of sneezing information, heart rate/respiratory rate information, and core temperature information of the object recognized by the biometric information recognition unit 154 and the device state information of the multi-object biometric information measurement device 100 as metadata.

The metadata generation unit 158 may transmit the metadata to the management server 200 through the communication module 130. The management server 200 may detect abnormal signs of disease based on the metadata.

The metadata generation unit 158 may generate sneezing metadata information based on the sneezing information, generate core temperature metadata information based on the core temperature information, and generate heart rate/respiratory rate metadata information based on the heart rate/respiratory rate information. In addition, the metadata generation unit 158 may generate device state metadata information based on the device state information that is able to confirm whether the multi-object biometric information measurement device 100 is operating normally. Here, the device may refer to the multi-object biometric information measurement device 100, and the device state information may include power on information about the moment when the power of the multi-object biometric information measurement device 100 is turned on, and alive information for determining whether the multi-object biometric information measurement device 100 is operating normally.

The power on information may include an event, device ID, time, etc. at the moment when power is applied to the multi-object biometric information measurement device 100 as shown in Table 1 below.

TABLE 1

| Key item | Value type | Description |
|---|---|---|
| event | String | Power_on |
| device_id | String | Device ID name |
| time | String | Year Month Day_Hour Minute Second |

Alive information is information for determining whether the multi-object biometric information measurement device 100 is operating normally, may be generated at a preset time cycle, and may include an event, a device ID, stream status, time, etc. as shown in Table 2 below.

TABLE 2

| | Value | |
|---|---|---|
| Key item | type | Description |
| event | String | alive |
| device_id | String | Device ID name |
| stream_status | String | “ok” or “error” |
| time | String | Year Month Day_Hour Minute Second |

Sneezing metadata information may include an event, device ID, sneeze/cough image, time, etc., as shown in Table 3 below. In this case, the sneeze/cough image may be in the form of a snapshot (image file).

TABLE 3

| Key item | Value type | Description |
|---|---|---|
| event | String | cough_info |
| device_id | String | Device ID name |
| image | Image | Image file |
| time | String | Year Month Day_Hour Minute Second |

Core temperature metadata information may include an event, device ID, radiant heat image, measured temperature, time, etc., as shown in Table 4 below. In this case, the radiant heat may be in the form of a snapshot (image file).

TABLE 4

| Key item | Value type | Description |
|---|---|---|
| event | String | thermal_info |
| device_id | String | Device ID name |
| image | Image | Image file |
| thermal | Float | Measured temperature |
| time | String | Year Month Day_Hour Minute Second |

Heart rate/respiratory rate metadata information may include an event, device ID, respiratory rate, heart rate, time, etc., as shown in Table 5 below.

TABLE 5

| Key item | Value type | Description |
|---|---|---|
| event | String | heart_info |
| device_id | String | Device ID name |
| respiratory_rate | Float | Respiratory rate |
| heart_rate | Float | Heart rate |
| time | String | Year Month Day_Hour Minute Second |

The metadata generation unit 158 may store metadata including at least one of the sneezing metadata information, heart rate/respiratory rate metadata information, core temperature metadata information, and device state metadata information in a specific directory folder. Then, the metadata generation unit 158 may automatically transmit the metadata to the management server 200 by an upload folder client program. Meanwhile, the processor 150 according to the embodiment of the present invention may further include an abnormal-signs-of-disease detection unit (not shown) that detects abnormal signs of disease of multiple objects in the measurement target space based on biometric information in the biometric information recognition unit 154 and predicts the disease occurrence risk. In this case, the abnormal-signs-of-disease detection unit may predict the disease occurrence risk by applying the biometric information to a disease prediction model (deep learning model).

The abnormal-signs-of-disease detection unit may transmit the predicted disease occurrence risk to the management server 200.

When the multi-object biometric information measurement device 100 configured as above is used, the abnormal signs of disease of multiple objects in an indoor space can be detected early, and the disease risk may be determined and shared quickly, thereby blocking the spread of a respiratory syndrome in advance.

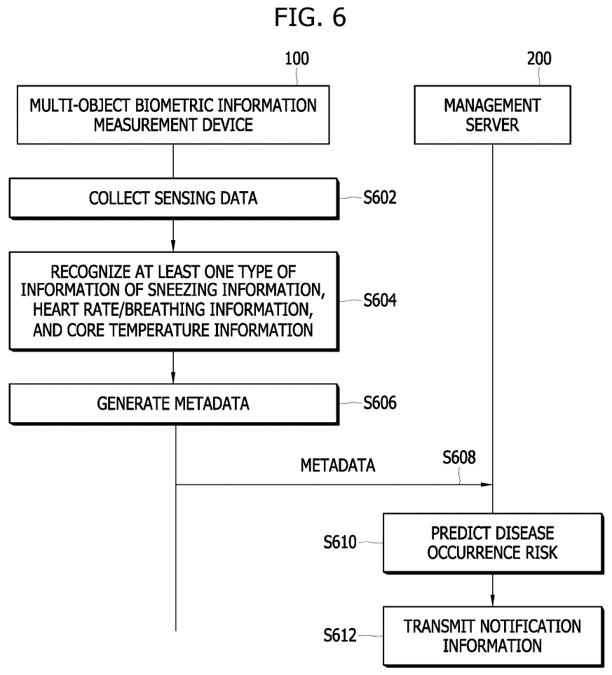

FIG. 6 is a diagram for describing a method of detecting abnormal signs of disease according to an embodiment of the present invention.

Referring to FIG. 6, the multi-object biometric information measurement device 100 collects sensing data through the sensor module 120 (S602). Here, the sensing data may include a thermal image, a real image, distance measurement information, an ambient temperature, etc.

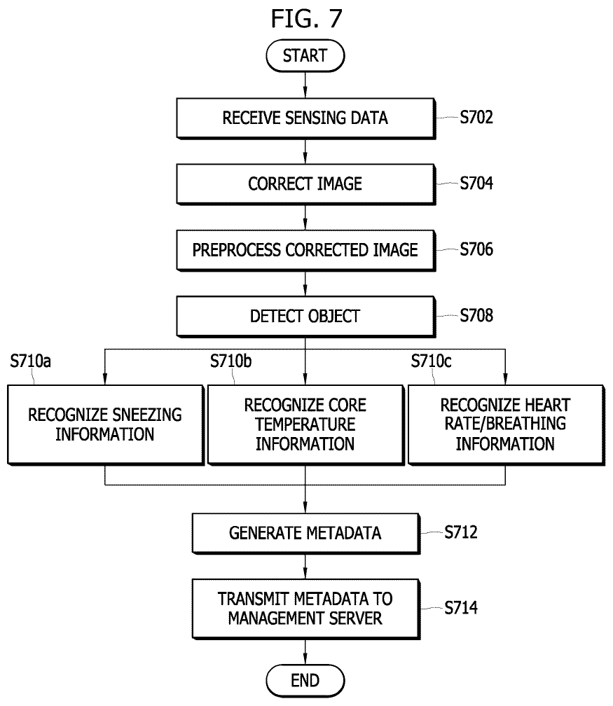

After operation S602 is performed, the multi-object biometric information measurement device 100 recognizes at least one of the sneezing information, heart rate/respiratory rate information, and core temperature information of an object located in a measurement target space based on the sensing data (S604). A detailed description of a method by which the multi-object biometric information measurement device 100 recognizes biometric information will be described with reference to FIG. 7.

After operation S604 is performed, the multi-object biometric information measurement device 100 generates metadata including biometric information and device state information (S606) and transmits the generated metadata to the management server (200 (S608). That is, the multi-object biometric information measurement device 100 may generate sneeze metadata information based on the sneezing information, generate core temperature metadata information based on the core temperature information, and generate heart rate/respiratory rate metadata information based on the heart rate/respiratory rate information. In addition, the multi-object biometric information measurement device 100 may generate device state metadata information based on the device state information that is able to confirm whether the multi-object biometric information measurement device 100 is operating normally.

After operation S608 is performed, the management server 200 predicts a disease occurrence risk based on the metadata from the multi-object biometric information measurement device 100 (S610), and transmits notification information including the predicted disease occurrence risk (S612). In this case, the management server 200 may store the metadata from the multi-object biometric information measurement device 100 and apply the stored metadata to the prediction model to predict a disease occurrence risk. Thereafter, the management server 200 may transmit notification information including the predicted disease occurrence risk to a user. In addition, the management server 200 may generate at least one of the sneeze metadata information, heart rate/respiratory rate metadata information, core temperature metadata information, and device state metadata information included in the metadata as notification information and transmit the generated notification information to an administrator.

FIG. 7 is a flowchart for describing a method of recognizing the biometric information of multiple objects according to an embodiment of the present invention.

Referring to FIG. 7, the processor 150 of the multi-object biometric information measurement device 100 receives sensing data including at least one of a thermal image, a real image, distance measurement information, and an ambient temperature from the sensor module 120 (S702).

After operation S702 is performed, the processor 150 corrects a thermal image and a real image so that the images match (S704). In this case, the processor 150 may correct the thermal image and the real image so that the images match by adjusting the intrinsic parameters of the first image sensor 122 that has acquired the thermal image and the second image sensor 124 that has acquired the real image. Through image matching, the object location of the thermal image and the object location of the real image may be matched.

After operation S704 is performed, the processor 150 performs preprocessing on the corrected real image (S706). In this case, the processor 150 may perform at least one preprocessing of adjusting the number of frames per second, converting the frame size, and data augmentation on the corrected real image.

After operation S706 is performed, the processor 150 detects an object corresponding to a preset class in the preprocessed real image (S708). In this case, the processor 150 may apply an object detection algorithm to the preprocessed real image to detect a person as an object.

After operation S708 is performed, the processor 150 recognizes sneezing information including the location, the number of times the object coughs, the number of times the object sneezes, etc. of an object that coughs/sneezes among the detected objects (S710a). In this case, the processor 150 may recognize the sneezing information by tracking the location of the object who coughs/sneezes, the number of times the object coughs and the number of times the object sneezes using a coughing/sneezing recognition model (behavior detection model) generated through a supervised learning technique based on the CNN.

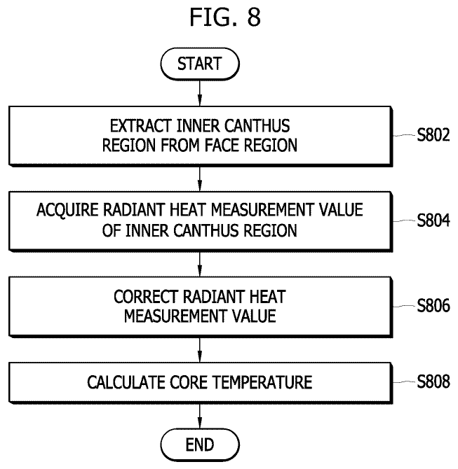

In addition, after operation S708 is performed, the processor 150 recognizes the core temperature information of the object based on a radiant heat measurement value of the detected object (S710b). In this case, the processor 150 may detect a specific region of the detected object, acquire a radiant heat measurement value of the specific region, and recognize the core temperature information of the object based on the radiant heat measurement value. The processor 150 may calculate the core temperature by applying the Biot Number concept. A detailed description of the method by which the processor 150 recognizes the core temperature information will be described with reference to FIG. 8.

In addition, after operation S708 is performed, the processor 150 recognizes heart rate/respiratory rate information based on the micro-Doppler signal, which is a signal generated when an antenna radiation beam formed by the distance measurement sensor 126 is reflected by the object and returned (S710c). Here, the micro-Doppler signal may be a signal with which periodic vibration (breathing, respiratory rate) information that may be detected from the front, side, and back of the object may be interpreted in the frequency domain.

Operations S710a, S710b, and S710c may be performed simultaneously.

After operation S710 is performed, the processor 150 generates biometric information including at least one of sneezing information, heart rate/respiratory rate information, and core temperature information of the recognized object and device state information of the multi-object biometric information measurement device 100 as metadata (S712), and transmits the generated metadata to the management server 200) (S714).

FIG. 8 is a flowchart for describing a method of calculating a core temperature according to an embodiment of the present invention.

Referring to FIG. 8, the processor 150 detects a face region from a detected object and extracts an inner canthus region from the detected face region (S802). In this case, the processor 150 may detect the face region of the object by applying a face detection algorithm to a thermal image or a real image, and extract the inner canthus region from the detected face region.

After operation S802 is performed, the processor 150 acquires a radiant heat measurement value of the inner canthus region (S804). In this case, the processor 150 may acquire the radiant heat measurement value based on a thermal image of the inner canthus region.

After operation S804 is performed, the processor 150 corrects the radiant heat measurement value based on distance data between the distance measurement sensor 126 and the object (or the inner canthus of the object) (S806). In this case, the processor 150 may correct the radiant heat measurement value by applying a compensation value determined in advance according to the distance.

After operation S806 is performed, the processor 150 calculates the core temperature using the corrected radiant heat measurement value, an atmospheric air temperature, and a head circumference of the object (S808). In this case, the processor 150 may calculate the core temperature by applying the head circumference, atmospheric air temperature, radiant heat measurement value, convection coefficient, and thermal conductivity of the object to the Biot number.

A multi-object biometric information measurement device, a system for detecting abnormal signs of disease including the same, and a method thereof according to some embodiments of the present invention have the effect of enabling early detection of abnormal signs of disease of multiple objects in the 3Cs (closed spaces/crowded places/close-contact settings) by measuring (recognizing) biometric information of the multiple objects in the in the 3Cs.

A multi-object biometric information measurement device, a system for detecting abnormal signs of disease including the same, and a method thereof according to some embodiments of the present invention have the effect of blocking the spread of diseases such as a respiratory syndrome in advance by detecting abnormal signs of disease early and predicting the disease risk based on biometric information collected from multi-object biometric information measurement devices installed nationwide, and informing the entire nation of the early detected abnormal signs of disease and the predicted disease risk.

Although the present invention has been described with reference to embodiments illustrated in the drawings, this is only exemplary, and those of ordinary skill in the art will understand that various modifications and other equivalent embodiments are possible therefrom. Therefore, the technical scope of protection of the present invention should be determined by the following patent claims.

What is claimed is:

1. A multi-object biometric information measurement device comprising:
at least one sensor module; and
a processor connected to the sensor module,
wherein the processor recognizes at least one of sneezing information, heart rate/respiratory rate information, and core temperature information of an object located in a measurement target space based on sensing data collected through the sensor module.

2. The multi-object biometric information measurement device of claim 1, wherein the sensor module includes:
a first image sensor that acquires a thermal image of the measurement target space;
a second image sensor that acquires a real image of the measurement target space;
a distance measurement sensor that measures a distance to the object; and
an environmental sensor that measures an atmospheric air temperature of the measurement target space.

3. The multi-object biometric information measurement device of claim 1, wherein the processor includes:
an object detection unit that detects an object corresponding to a preset class in a thermal image acquired through a first image sensor and a real image acquired through a second image sensor; and
a biometric information recognition unit that recognizes biometric information including at least one of sneezing information, heart rate/respiratory rate information, and core temperature information of the object detected by the object detection unit.

4. The multi-object biometric information measurement device of claim 3,
wherein the processor further includes an image correction unit that corrects the thermal image and the real image so that the images match, and
the object detection unit generates a bounding box of the detected object to perform object tracking when the object corresponding to the preset class is present in the corrected real image.

5. The multi-object biometric information measurement device of claim 3, wherein the biometric information recognition unit recognizes the sneezing information by applying the detected object to a behavior detection model to determine whether the object is coughing/sneezing, and counting the number of times the object coughs/sneezes while tracking the object that coughs or sneezes.

6. The multi-object biometric information measurement device of claim 3, wherein the biometric information recognition unit detects a specific region in the detected object, acquires a radiant heat measurement value of the specific region, and calculates a core temperature of the object based on the radiant heat measurement value.

7. The multi-object biometric information measurement device of claim 6, wherein the biometric information recognition unit calculates the core temperature by applying a compensation value set according to a distance between a distance measurement sensor and an object to the radiant heat measurement value to correct the radiant heat measurement value and applying the corrected radiant heat measurement value, an atmospheric air temperature measured through an environmental sensor and a head circumference of an object to the Biot number.

8. The multi-object biometric information measurement device of claim 6, wherein the biometric information recognition unit recognizes a heart rate and respiratory rate of the object by analyzing a micro-Doppler signal, which is a signal generated when an antenna radiation beam formed by a distance measurement sensor is reflected by the object and returned.

9. The multi-object biometric information measurement device of claim 3, wherein the processor further includes a metadata generation unit that generates biometric information including at least one of the recognized sneezing information, heart rate/respiratory rate information, and core temperature information of the object, and device state information of the multi-object biometric information measurement device as metadata, and transmits the generated metadata to an external device through a communication module.

10. The multi-object biometric information measurement device of claim 3, wherein the processor further includes an abnormal-signs-of disease detection unit that detects abnormal signs of disease of multiple objects within the measurement target space based on the biometric information recognized by the biometric information recognition unit, and predicts a disease occurrence risk.

11. A system for detecting abnormal signs of disease, comprising:
a plurality of multi-object biometric information measurement devices, each of which is installed in one measurement target space and recognizes at least one type of biometric information among sneezing information, heart rate/respiratory rate information, and core temperature information of an object that is present in the corresponding measurement target space, and transmit metadata including the biometric information and device state information to a management server; and
the management server that predicts a disease occurrence risk based on the metadata from the plurality of multi-object biometric information measurement devices.

12. The system of claim 11, wherein the management server generates notification information including the predicted disease occurrence risk and notifies users of the notification information.

13. A method of detecting abnormal signs of disease, comprising:
receiving, by a processor, sensing data including at least one of a thermal image, a real image, distance measurement information, and an ambient temperature from a sensor module;
detecting, by the processor, an object corresponding to a preset class in the thermal image and the real image; and
recognizing, by the processor, biometric information including at least one of sneezing information, heart rate/respiratory rate information, and core temperature information of the detected object.

14. The method of claim 13, wherein, in the detecting of the object, the processor corrects the thermal image and the real image so that the images match and generates a bounding box of the detected object to perform object tracking when the object corresponding to the preset class is present in the corrected real image.

15. The method of claim 13, wherein, in the recognizing of the biometric information, the processor recognizes the sneezing information by applying the detected object to a behavior detection model to determine whether the object is coughing/sneezing, and counting the number of times the object coughs/sneezes while tracking the object that coughs or sneezes.

16. The method of claim 13, wherein, in the recognizing of the biometric information, the processor detects a specific region in the detected object, acquires a radiant heat measurement value of the specific region, and calculates a core temperature of the object based on the radiant heat measurement value.

17. The method of claim 16, wherein, in the recognizing of the biometric information, the processor calculates the core temperature by applying a compensation value set in the distance measurement information and an object to the radiant heat measurement value to correct the radiant heat measurement value and applying the corrected radiant heat measurement value, an atmospheric air temperature measured through an environmental sensor, and a head circumference of an object to the Biot number.

18. The method of claim 13, wherein, in the recognizing of the biometric information, the processor recognizes a heart rate and respiratory rate of the object by analyzing a micro-Doppler signal, which is a signal generated when an antenna radiation beam formed by a distance measurement sensor is reflected by the object and returned.

19. The method of claim 13, further comprising, after the recognizing of the biometric information, generating, by the processor, metadata including the biometric information and device state information and transmitting the metadata to a management server.

20. The method of claim 13, further comprising, after the recognizing of the biometric information, detecting, by the processor, abnormal signs of disease of multiple objects within a measurement target space based on the recognized biometric information and predicting a disease occurrence risk.
